(19) United States
(12) Patent Application Publication
(10) Pub. No.: US 2011/0190541 A1
(43) Pub. Date: Aug. 4, 2011

(54) METHOD FOR THE SYNTHESIS OF HIGH PURITY PRIMARY DIAMINES AND/OR TRIAMINES

(75) Inventors: Thierry Beillon, Sainte Foy Les Lyon (FR); Jean-Philippe Gillet, Brignais (FR)

(73) Assignee: Ceca S.A., La Garenne Colombes (FR)

(21) Appl. No.: 12/446,323

(22) PCT Filed: Oct. 26, 2007
(86) PCT No.: PCT/FR07/52253

§ 371 Date: Oct. 4, 2010

(30) Foreign Application Priority Data
Oct. 27, 2006 (FR) 0654595
Apr. 5, 2007 (FR) 0754304

(51) Int. Cl. C07C 209/48

(52) U.S. Cl. 564/491

(57) ABSTRACT

The present invention relates to a process for the preparation of primary di- and/or triamines of high purity from nitriles which can themselves originate from dimer and/or trimer acids.

This process comprises a stage of ammoniation of the acid functional groups and a stage of hydrogenation of the nitrile functional groups to give primary amine functional groups and does not require additional purification stage(s).

### TECHNICAL FIELD

The present invention relates to a process for the synthesis of primary diamines and/or triamines from dimer and/or trimer nitriles, it being possible for these nitriles themselves to originate from dimer and/or trimer fatty acids.

These amines have numerous applications as corrosion inhibitors, in detergency, as additives for bitumen, flotation agents, anticaking agents, antidust agents, crosslinking agents, oil additives, lubricating agents, additives in water treatment or additives for concrete.

### STATE OF THE PRIOR ART

Diamines and triamines from dimer and trimer fatty acids have been known since the 1950s and have an EINECS number and are described, for example, by the Kirk-Othmer Encyclopedia, 4th edition, vol. 8, chapter Dimer Acids (pages 223 to 237).

Dimer and trimer acids are obtained by polymerization, at high temperatures and under pressure, of unsaturated fatty acids. These unsaturated fatty acids, predominantly oleic (C:18-1) or linoleic (C:18-2) acids, essentially originate from tall oil, which itself results from paper pulp processes of kraft type. This source of acid is favored for reasons of cost (85% of the acids consumed in this field) but it is entirely possible to use unsaturated fatty acids originating from other plant sources.

After polymerization of these acids, a mixture is obtained which comprises, on average, 30-35% of monocarboxylic acids, often isomerized with respect to the starting acids, 60-65% of dicarboxylic acid (dimer acids) with the double carbon number with respect to the starting acids and 5-10% of tricarboxylic acids (trimer acids) having the triple carbon number with respect to the starting acids. By purifying this mixture, the various commercial grades of dimer acids or trimer acids, which can exist in the hydrogenated or non-hydrogenated form, are obtained.

Mention may be made, among these, of the Pripol range developed by Unichema. These products are compounds of choice in numerous applications by virtue of their properties, such as high hydrophobicity, good stability with regard to heat, UV radiation and oxygen, and good compatibility with the materials.

The major advantage of diacids and triacids lies in the fact that these compounds remain liquid at ambient temperature while having a low viscosity, despite their mean carbon number of 36 or 54. This is due to the mixture of the numerous isomers of which the product is composed and also to the cycloaliphatic rings and to the presence of unsaturations. Furthermore, the majority of diacids and triacids result from plant raw materials and are thus renewable.

The synthesis of these amines from fatty acids which are first di- or trimerized takes place in two stages: conversion of the carboxyl functional groups to nitrite functional groups by reaction of ammonia in the presence of a catalyst and then conversion of the nitrile functional groups to amine functional groups in the presence of a hydrogenation catalyst, in order to obtain amines. For example, U.S. Pat. No. 2,526,044 describes (column 4, line 62) that the polynitriles obtained from castor oil fatty acids dehydrated in the presence of phosphorus can be hydrogenated to give polyamines by means of nickel or platinum catalyst. However, beforehand, the polynitrile has to be distilled, despite a very high boiling point.

U.S. Pat. No. 3,010,782 describes (column 1, line 40) the synthesis of polynitriles from octadecadienoic acid and ammonia which can subsequently be hydrogenated to give polyamines but without specifying their degree of purity.

U.S. Pat. No. 3,231,545 discloses (column 2, line 61) that dimer fatty acids can be converted to the corresponding nitriles and then hydrogenated to give diamines. Furthermore, it is specified that a purification is necessary at each stage in order to obtain dimers of good purity allowing them to be used in the field of polymers.

These same indications are given in U.S. Pat. No. 3,242,141 and U.S. Pat. No. 3,483,237; in the latter patent, it is additionally specified (column 5, line 74) that the hydrogenation as described results in a diamine comprising a high level of secondary and tertiary amine.

The need to purify the products resulting from each of the stages is also mentioned in U.S. Pat. No. 3,475,406, where it is specified that these diamines have to be purified by distillation in order for the level of impurities to be less than 10% and preferably less than 5% (column 5, line 35).

The teaching of all these patents is that it is necessary to purify the nitriles before their conversion to amines and/or that it is necessary to purify the amines on conclusion of the process in two stages by distillation, which is particularly difficult given the boiling point of these products.

### DESCRIPTION OF THE INVENTION

The present invention provides first of all a process for the synthesis of high-purity di- and/or triamines from di- or trinitriles (also known subsequently as “the nitriles”) by hydrogenation.

The di- and/or trinitriles employed can in particular be mixtures of dimerization and/or trimerization products of mononitriles generally comprising 8 to 30 carbon atoms and one or more unsaturations, mainly in the form of double bond(s), which allow said dimerization and/or trimerization.

This stage of hydrogenation of the nitriles to give primary amines takes place in a reactor under pressure, for example in an autoclave, in the presence of a hydrogenation catalyst, of ammonia and optionally of at least one strong base. The nitriles and the hydrogenation catalyst, such as, for example, Raney nickel, Raney cobalt, palladium supported on charcoal or alumina or rhodium supported on charcoal or alumina are charged to the reactor, which is subsequently purged with nitrogen.

The ammonia is subsequently introduced at ambient temperature, so as to create an ammonia partial pressure, and the reaction medium is brought with stirring to a temperature of between 100° C. and 130° C. before introducing the hydrogen. The reaction temperature is generally, in the broad sense, between 110° C. and 170° C. and preferably from 130° C. to 150° C.

The amount of hydrogenation catalyst employed represents from 0.1% to 15% by weight, preferably from 3% to 10% by weight, of the charge of the nitriles and more preferably 4% to 8% by weight.

The total pressure of the reactor during this stage is generally between 2 MPa and 4 MPa but it would be possible to operate at a higher pressure (15 MPa) without disadvantage and without departing from the scope of the invention.

The reaction can be carried out in a solvent-comprising medium, the solvent being chosen from conventional solvents used for this type of reaction.

According to an advantageous embodiment, the reaction is carried out in the absence of solvent, in particular in the case where the starting polynitriles are in the liquid form.

The reaction is continued in this way until hydrogen consumption has ceased and until the measurement of the basicity number no longer varies.

In the context of the present invention, the ammonia/nitrile functional groups molar ratio is between 0.2 and 3.

The term “ammonia/nitrile functional groups molar ratio” is understood to mean the ratio of the number of moles of ammonia introduced to the number of nitrile functional groups present in the reaction medium.

The number of nitrile functional groups present in the reaction medium can be determined by any quantitative analytical method known to a person skilled in the art and for example by quantitative analysis by infrared spectrometry.

When the polynitrile involved in the hydrogenation reaction originates from a mixture of fatty acids as indicated above, it is possible to envisage quantitatively determining the number of acid functional groups according to techniques known to a person skilled in the art. The number of nitrile functional groups generated during the ammoniation reaction described later can then be understood as being equal to the number of acid functional groups converted.

It has been discovered, surprisingly, and it is this which forms one of the aspects of the present invention, that the addition of a relatively small amount of base to the reaction medium for the hydrogenation of the nitrile functional groups to give amine functional groups makes it possible to substantially reduce the amount of ammonia introduced while retaining the selectivity which would be obtained with a greater amount of ammonia.

The base which can be added to the reaction medium can be of any type and in particular a strong organic or inorganic base, preferably a strong inorganic base, in particular chosen from alkali metal or alkaline earth metal hydroxides, for example sodium hydroxide or potassium hydroxide. Preference is given in particular to the use of sodium hydroxide. A mixture of two or more bases can also be used.

Thus, when the ammonia/nitrile functional groups molar ratio is between 0.2 and 1.3 and preferably between 0.5 and 1, at least one strong base, such as sodium hydroxide and/or potassium hydroxide, is added to the reaction mixture in a proportion of 0.07 to 1 mol % and preferably of 0.35 to 0.75 mol %, with respect to the number of nitrile functional groups present in the reaction medium and as were defined above. The at least one strong base is preferably added in the aqueous form. It should be understood that, when the ammonia/nitrile functional groups molar ratio is between 1.3 and 3 and preferably between 1.5 and 2.6, the presence of strong base may be dispensed with.

The hydrogenation stage of the process according to the invention makes it possible to 100% convert the nitrile functional groups to primary amine functional groups with a selectivity for primary amines of greater than 97%, which makes it possible to use the diamines and triamines directly and without purification in the applications where the required purity is very high.

The polynitriles, in particular di- and trinitriles, employed in the process for the preparation of primary amines, mainly in the form of diamines and triamines, can advantageously be obtained from di- and/or trimer fatty acids according to conventional ammoniation techniques known to a person skilled in the art.

The ammoniation reaction can, for example, be carried out conventionally in the presence of an ammoniation catalyst preferably chosen from metal oxides, preferably zinc oxide, in a catalyst/diacids and/or triacids ratio by weight of between 0.01% and 0.15% and preferably 0.03% and 0.1%. The reaction medium is placed under stirring and brought to a temperature generally ranging from 150° C. to 170° C., then gaseous ammonia is introduced into the reactor, for example using a dip pipe, and the temperature is increased, preferably stepwise, to a temperature generally ranging from 250° C. to 320° C., preferably from 290° C. to 310° C. The pressure is generally between 0.05 MPa and 0.4 MPa, atmospheric pressure (0.1 MPa) being preferred. The water formed and the excess ammonia can be collected in a trap via a dephlegmator maintained at 130° C. The reaction is continued until the acid number of the reaction medium is less than or equal to 0.1 mg KOH/g, i.e. a time of 12 to 17 hours. The mass spectroscopy and infrared analyses show that the acid functional groups are converted virtually quantitatively to nitriles.

As for the hydrogenation reaction described above, the ammoniation reaction can be carried out in a solvent-comprising medium. However, it is preferable to carry out the conversion of the acid functional groups to nitrile functional groups in the absence of solvent, in particular when the compounds carrying acid functional groups are employed in the liquid state.

The nitriles thus obtained can be used as is, that is to say without intermediate purification, in the hydrogenation reaction described above to form the di- and triamines.

According to another aspect, the present invention provides a process for the synthesis of high-purity di- and/or triamines from di- and/or trimer fatty acids in two stages which does not require any purification stage, comprising the following stages:

A) in a reactor with stirring, conversion of the acid functional groups of the dimer and/or trimer acids to nitrile functional groups, in order to obtain di- and trinitriles, in the presence of an ammoniation catalyst preferably chosen from metal oxides, preferably zinc oxide, in a catalyst/diacids and/or triacids ratio by weight of between 0.01% and 0.15%, then introduction of gaseous ammonia into the reactor,
B) in a reactor under pressure, conversion of the nitrile functional groups of the product resulting from stage A) to primary amine functional groups by employing the process described above, that is to say by hydrogenation, in the presence of a hydrogenation catalyst and hydrogen, in which conversion, after bringing the nitriles and the hydrogenation catalyst into contact, the ammonia is introduced at ambient temperature and the reaction medium is brought with stirring before introducing the hydrogen, the reaction temperature ranging from 110° C. to 170° C. and preferably from 130° C. to 150° C.,
the amount of hydrogenation catalyst employed represents from 0.1% to 15% by weight of the charge of nitriles, and
the ammonia/nitrile functional groups molar ratio is between 0.2 and 3.

In the 1st stage (stage A), the acid functional groups of the dimer and/or trimer acids are converted to nitrile functional groups in order to obtain di- and trinitriles (ammoniation reaction described above) and, in the second stage (stage B), the nitrile functional groups are converted to primary amine functional groups by hydrogenation, as indicated above.

In particular, the process of the invention can advantageously be employed in the preparation of primary amines, in the form of di- and/or triamines of high purity, with high selectivity. The term “high selectivity” is understood to mean that the nitrile functional groups are converted to primary amine functional groups, in particular converted to primary amine functional groups at more than 95%, with respect to the total number of amine functional groups formed, more specifically to primary amine functional groups at more than 97%. The other amine functional groups formed may be predominantly secondary amines, for example in proportions of less than 5%, preferably of less than 3%, with respect to the total number of amine functional groups formed. With regard to the tertiary amines, if they are formed, they are generally only hi the form of traces.

The process of the present invention has an entirely advantageous application in the selective synthesis of primary di- and/or triamines with high selectivity from unsaturated fatty acids originating from tall oil or other plant sources and which are mainly in the form of di- and/or trimers. Such acid forms are well known and are described, for example, in patent U.S. Pat. No. 3,475,406 or also patent application WO 2003/054092.

The process for the synthesis of primary di- and/or triamines from unsaturated fatty acids can be represented according to the following scheme:

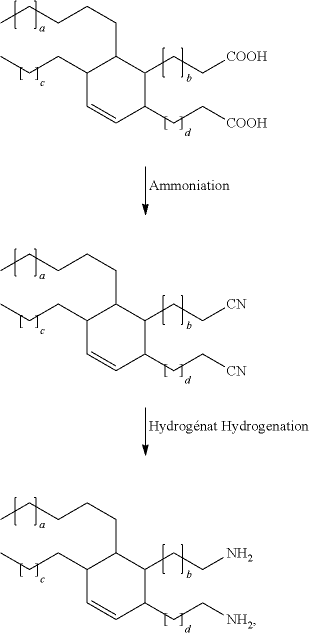

in which scheme only diacids, dinitriles and diamines are represented and a, b, c and d represent, independently of one another, the number of methylene (—CH₂—) links in each of the chains. Generally, a, b, c and d are each between 1 and 24, more generally between 2 and 20, more particularly between 4 and 16.

Due to their great purity and their high selectivity (>95% primary amines), the primary amines obtained according to the process of the present invention have applications in a great many fields. Mention may be made, as examples of use of these amines, of their use as corrosion inhibitors, in detergency, as additives for bitumen, flotation agents, anticaking agents, antidust agents, crosslinking agents, oil additives, lubricating agents, additives in water treatment, additives for concrete, and others.

The examples which follow are provided by way of illustration of the present invention without introducing any limiting nature on the scope of the protection defined by the claims appended to the present description.

### Example 1

### Synthesis of a Dinitrile from Pripol 1013

2516 g of dimerized fatty acid, sold under the name Pripol 1013 and having an acidity number of 191.9 mg of KOH/g, are charged to a predried 31 glass reactor equipped with a mechanical stirrer, electrical heating, a dephlegmator, a reflux condenser and a dry ice trap, and a system for introducing ammonia. A catalytic charge of zinc oxide of 1.57 g, i.e. 0.0625% of the weight of dimerized fatty acid employed, is added. The reaction medium is placed under stirring and then heated up to 160° C. Gaseous ammonia is then introduced at the rate of 0.417 l/min·kg. The reaction medium is brought to 300° C. The introduction of ammonia is continued until the acidity number of the reaction medium is less than 0.1 mg of KOH/g. The reaction time is approximately from 12 to 14 h. At the end of, the reaction, the reaction medium is cooled to 40° C. and the reactor is emptied. The yield is in the region of 100% and the selectivity for dinitrile is virtually 100%.

### Example 2

### Synthesis of a Dinitrile from Pripol 1048

2130 g of dimer/trimer fatty acid sold under the name Pripol 1048 (hydrogenated dimer and trimer acid mixture) and having an acidity number of 187.8 mg of KOH/g are charged to an installation identical to that of example 1. A catalytic charge of zinc oxide of 1.33 g, i.e. 0.0625% of the weight of fatty acid employed, is added. The reaction medium is placed under stirring and then heated up to 160° C. Gaseous ammonia is then introduced at the rate of 0.417 l/min·kg. The reaction medium is brought to 300° C. The introduction of ammonia is continued until the acidity number of the reaction medium is less than 0.1 mg of KOH/g. The reaction time is 15 h. At the end of the reaction, the reaction medium is cooled to 40° C. and the reactor is emptied. The yield is in the region of 100% and the selectivity for the nitrile functional groups is virtually 100%.

### Example 3

### Synthesis of a Diamine from Pripol 1013

200 g of dinitrile resulting from example 1 (Pripol 1013) and 15 g of Raney nickel, filtered off and washed with isopropanol, i.e. 7.5% by weight of the initial dinitrile charge, are charged to a 500 cm³ autoclave. The reactor is closed under pressure, a check is carried for leaktightness and the reactor is rendered inert with nitrogen by compression/decompression. The gaseous ammonia is subsequently introduced at ambient temperature, which gives a pressure of 0.5 to 0.6 MPa at 25° C. This corresponds in this case to a weight from approximately 25 to 35 g of anhydrous ammonia. The reaction medium is brought to 120-130° C. with stirring and then hydrogen is introduced in order to have a total pressure of 2.3 to 2.5 MPa. Consumption of hydrogen is immediate. Monitoring is provided by measurement of the basicity as the reaction progresses. The latter lasts in the vicinity of 12 hours. At the end of the reaction, the reaction medium is cooled to ambient temperature, the hydrogen and the ammonia are purged with nitrogen and then the crude reaction product is emptied out. The catalyst is recovered by filtering under nitrogen and can be recycled. The conversion of the nitrile is 100% and the content of secondary amines is less than 3% (NMR quantification limit).

### Example 4

### Synthesis of a Diamine from Pripol 1048

200 g of nitrile resulting from example 2 (from Pripol 1048) and 15 g of Raney nickel, filtered off and washed with isopropanol, i.e. 7.5% by weight of the initial charge of nitrile from Pripol 1048, are charged to a 500 cm³ autoclave. The reactor is closed under pressure, a check is carried out for leaktightness and the reactor is rendered inert with nitrogen by compression/decompression. The gaseous ammonia is subsequently introduced at ambient temperature, which gives a pressure of 0.6 MPa at 25° C. The reaction medium is brought to 120-130° C. with stirring and then hydrogen is introduced in order to have a total pressure of 2.5 MPa. Consumption of hydrogen is immediate. Monitoring is provided by measurement of the basicity as the reaction progresses. The reaction lasts 12 h. At the end of the reaction, the reaction medium is cooled to ambient temperature, the hydrogen and the ammonia are purged with nitrogen and then the crude reaction product is emptied out. The catalyst is recovered by filtering under nitrogen and can be recycled. The conversion of the nitrile is 100% and the content of secondary amines is less than 3% (NMR quantification limit).

### Examples 5 to 12

### Synthesis of Diamines from Pripol 1013

Other amines were synthesized from the dinitrile from Pripol 1013 of example 1; the second stage was carried out with different operating conditions from those of the preceding example 3 or 4 (level and nature of catalyst, ammonia partial pressure, possible presence of water in the catalyst, possible addition of strong base). The operating conditions of examples 5 to 12 and also the characteristics of the diamines synthesized are given in detail in the table below:

| | Comparative example 5 | Comparative example 6 | Comparative example 7 | Example 8 | Example 9 |
|---|---|---|---|---|---|
| Catalyst 2nd stage | Raney Ni, washed and | Raney Ni, washed and | Raney Ni, washed and | Raney Ni + H₂O | Raney Ni |
| | filtered off | filtered off | filtered off | | |
| Amount (g) | 6.2 | 20 | 10 | 15 + 2.8 | 10 |
| % with respect to the nitrile | 2 | 10 | 5 | 7.5 | 5 |
| Nitrile (g) | 310 | 200 | 200 | 200 | 200 |
| Ammonia pressure NH₃ (MPa) | 0.7 at 65° C. | 0.7 at 65° C. | 0.7 at 65° C. | 0.56 at 25° C. | 0.56 at 25° C. |
| Amount (g) | 11.5 | 11.5 | 11.5 | 31.2 | 28.6 |
| Total pressure (MPa) | 2.3 | 2.3 | 2.3 | 2.3 | 2.3 |
| Temperature (° C.) | 120-130 | 130-150 | 145-150 | 130 | 130 |
| Duration (h) | 27 | 11 | 10 | 10 | 12 |
| Final alkalinity (mg of KOH/g) | 3.02 | 3.29 | 3.31 | 3.48 | 3.39 |
| NMR analyses (initial mol %) | | | | | |
| CN | 7.6 | 0 | 0 | 0 | 0 |
| NH₂ (amine I) | 92.4 as amines | 75 | 78 | >97 | >97 |
| NH (amine II) | (I + II) | 25 | 22 | traces (<3) | traces (<3) |

| | Example 10 | Example 11 | Example 12 |
|---|---|---|---|
| Catalyst 2nd stage | Raney Ni | Raney Ni | Raney Co |
| Amount (g) | 15 | 10 | 15 |
| % with respect to the nitrile | 7.5 | 5 | 7.5 |
| Strong base | NaOH | NaOH | NaOH |
| Mol %/nitrile functional groups | 0.68 | 0.68 | 0.68 |
| Nitrile (g) | 200 | 200 | 200 |
| Ammonia/nitrile functional groups molar ratio | 0.92 | 0.9 | 0.92 |
| Ammonia pressure NH₃ (MPa) | 0.56 at 50° C. | 0.56 at 50° C. | 0.56 at 50° C. |
| Amount (g) | 11.5 | 11.5 | 11.5 |
| Total pressure (MPa) | 2.3 | 2.3 | 2.3 |
| Temperature (° C.) | 130 | 130 | 130 |
| Duration (h) | 10 | 10 | 10 |
| Final alkalinity (mg of KOH/g) | 3.5 | 3.45 | 3.55 |
| NMR analyses (initial mol %) | | | |
| CN | 0 | 0 | 0 |
| NH₂ (amine I) | >97 | >97 | >97 |
| NH (amine II) | <3 | <3 | <3 |

### Example 13

### Synthesis of the Dinitrile from Azelaic Acid

2000 g (10.63 mol) of azelaic acid and 1.25 g of zinc oxide, i.e. 0.0625% by weight with respect to the azelaic acid, are charged to a 4 l glass reactor equipped with a dephlegmator, a mechanical stirrer, a system for introducing gaseous ammonia and an electrical heating system.

The reaction medium is brought to 130° C. so as to melt the diacid. Stirring is begun and the temperature is brought to 210° C. The ammonia is then gradually introduced up to a nominal flow rate of 0.417 l/min and per kg. The temperature of the reaction medium is raised to 290-300° C. The temperature of the dephlegmator is 130° C. The progress of the reaction is monitored by the acidity number of the reaction medium. After 17 hours, the ammonia flow is halted and the reaction medium is cooled. The latter is subsequently distilled under reduced pressure and an azelonitrile is obtained with a purity of 99% and a yield of 85%.

### Example 14

### Synthesis of 1,9-diaminononane with ammonia and strong base

300 g (2 mol) of azelonitrile obtained in example 13 are charged, with 9 g of Raney nickel, to a clean and dry 500 cm³ autoclave. This autoclave is closed and the gas phase is purged with nitrogen. 17 g of ammonia (1 mol, i.e. 0.25 mol of NH₃/mole of CN functional group) and 0.6 g of 50% by weight sodium hydroxide in water are subsequently introduced at ambient temperature. The reaction medium is placed under stirring and then the hydrogen is introduced so that the total pressure is 30 bar at 130° C.

After reacting for 6 hours, cooling is carried out and the catalyst is filtered off at a temperature of 60° C. The crude diamine is distilled conventionally under reduced pressure. The 1,9-diaminononane is obtained with a purity of 99.2% and a yield of 88%. The diamine does not comprise impurity such as ethyl-1,9-diamino-nonane.

### Example 15

### Synthesis of 1,10-diaminodecane with ammonia alone

300 g (1.83 mol) of sebaconitrile are charged, with 9 g of Raney nickel, to a clean and dry 500 cm³ autoclave. The autoclave is closed and the gas phase is purged with nitrogen. 50 g of ammonia (2.94 mol, i.e. 0.8 mol of NH₃/mole of CN functional group) are subsequently introduced at ambient temperature. The reaction medium is placed under stirring and then the hydrogen is introduced so that the total pressure is 30 bar at 130° C.

After reacting for 19 hours, cooling is carried out and the catalyst is filtered off at a temperature of 80° C. The crude diamine is distilled conventionally. The 1,10-decanediamine is obtained with a purity of 99% and a yield of 85%.

### Example 16

### Synthesis of 1,10-diaminodecane with ammonia and strong base

300 g (1.83 mol) of sebaconitrile are charged, with 9 g of Raney nickel, to a clean and dry 500 cm³ autoclave. The latter is closed and the gas phase is purged with nitrogen. 15 g of ammonia (0.88 mol, i.e. 0.24 mol of NH₃/mole of CN functional group) and 0.6 g of 50% by weight sodium hydroxide in water are subsequently introduced at ambient temperature. The reaction medium is placed under stirring and then the hydrogen is introduced so that the total pressure is 30 bar at 130° C.

After reacting for 6 hours 30 minutes, cooling is carried out and the catalyst is filtered off at a temperature of 80° C. The crude diamine is distilled conventionally. The 1,10-decanediamine is obtained with a purity of 99.3% and a yield of 90%. The diamine does not comprise ethyl-1,10-diaminodecane.

### Example 17

### Synthesis of 1,10-diaminodecane with ammonia, strong base and solvent

150 g (0.914 mol) of sebaconitrile are charged, with 4.5 g of Raney nickel and 150 g of ethanol, to a clean and dry 500 cm³ autoclave. The latter is closed and the gas phase is purged with nitrogen. 35.2 g of ammonia (2.07 mol, i.e. 1.13 mol of NH₃/mole of CN functional group) and 0.3 g of 50% by weight sodium hydroxide in water are subsequently introduced at ambient temperature. The reaction medium is placed under stirring and then the hydrogen is introduced so that the total pressure is 30 bar at 130° C.

After reacting for 5 hours, cooling is carried out and the catalyst is filtered off at a temperature of 30° C. The solvent is evaporated and then the crude diamine is distilled conventionally. The 1,10-decanediamine is obtained with a purity of 98.5% and a yield of 90%. The diamine comprises ethyl-1,10-diaminodecane.

1-3. (canceled)

4. The process as claimed in claim 12, in which the reaction is carried out at a total pressure of between 2 MPa and 15 MPa.

5. The process as claimed in claim 12, in which the amount of hydrogenation catalyst employed represents 3% to 10% by weight of the charge of nitrile functional groups and in that the molar ratio of ammonia functional groups to nitrile functional groups is between 0.5 and 1.

6. The process as claimed in claim 12, characterized in that the amount of hydrogenation catalyst employed represents 4% to 8% by weight of the charge of nitrile functional groups and in that the of ammonia functional groups to nitrile functional groups is between 1.5 and 2.6.

7. The process as claimed in claim 12, characterized in that the hydrogenation catalyst is chosen from Raney nickel, Raney cobalt, palladium supported on charcoal or alumina or rhodium supported on charcoal or alumina.

8-11. (canceled)

12. A process for the synthesis of di- and/or triamines from di- or tri-nitriles comprising the conversion of the nitrile functional groups of the di- or tri-nitriles to primary amine functional groups by hydrogenation in the presence of a hydrogenation catalyst and hydrogen comprising the steps of:
bringing the di- or tri-nitriles into contact with the hydrogenation catalyst;
introducing ammonia at ambient temperature to form a reaction medium;
stirring the reaction medium; and thereafter
introducing hydrogen,
wherein the process temperature ranges from 110° C. to 170° C., the amount of hydrogenation catalyst employed represents from 0.1% to 15% by weight of the di- or tri-nitriles, and the molar ratio of ammonia functional groups to nitrile functional groups is between 0.2 and 3.

13. The process of claim 12 wherein the process temperature ranges from 130° C. to 150° C.

14. The process as claimed in claim 12, in which, when the molar ratio of ammonia functional groups to nitrile functional groups is between 0.2 and 1.3, at least one strong base is added to the reaction medium in a proportion of 0.07 to 1 mol % with respect to the nitrile functional groups.

15. The process of claim 14 wherein the molar ratio of ammonia functional groups to nitrile functional groups is between 0.5 and 1

16. The process of claim 14 wherein the at least one strong base is in aqueous form,

17. The process of claim 14 wherein the at least one strong base is added to the reaction medium in a proportion of 0.35 to 0.75 mol %.

18. The process as claimed in claim 17, in which, when the molar ratio of ammonia functional groups to nitrile functional groups is between 1.3 and 3, the presence of strong base is optional.

19. The process of claim 18 wherein the molar ratio of ammonia functional groups to nitrile functional groups is between 1.5 and 2.6.

20. The process of claim 12 in which the reaction is carried out at a total pressure of between 2 MPa and 4 Mpa.

21. A process for the synthesis of di- and/or tri-amines from di- and/or trimer fatty acids, comprising the steps of:
A) reacting, with stirring, the acid functional groups of the dimer and/or trimer acids to nitrite functional groups with gaseous ammonia, in the presence of an ammoniation catalyst to obtain di- and tri-nitriles wherein the weight ratio of ammoniation catalyst to di- and/or trimer fatty acids is between 0.01% and 0.15%; and
B) converting the di- and tri-nitriles resulting from step A) to primary amine functional groups via the process as claimed in claim 12.

22. The process of claim 21 wherein the reaction of step A) starts at a pressure of between 0.05 and 0.4 Mpa, at a temperature ranging from 150° C. to 170° C. and is thereafter increased stepwise to a temperature ranging from 250° C. to 320° C.

23. The process of claim 22, wherein the pressure is atmospheric pressure.

24. The process of claim 22, wherein the temperature is increased to a temperature of from 290° C. to 310° C.

25. The process of claimed in claim 21, characterized in that in step A), the ratio, by weight, of catalyst to dimer and/or trimer acids is between 0.03% and 0.1%.

26. The process of claim 21 wherein the ammoniation catalyst is a metal oxide.

27. The process of claim 23 wherein the metal oxide is zinc oxide.
